(19) United States
(12) Patent Application Publication
(10) Pub. No.: US 2005/0241590 A1
(43) Pub. Date: Nov. 3, 2005

(54) Metamorphosizing gift item and method of gift-giving which utilizes the life-cycle of a living being

(76) Inventors: Ann M. Koznarek, Hinsdale, IL (US); Marco B. Imburgia, Schaumburg, IL (US)

Correspondence Address: LAW OFFICE OF BRIAN R. RAYVE, P.O. Box 11586, Salt Lake City, UT, 84147-0586, US

(21) Appl. No.: 10/835,884

(22) Filed: Apr. 30, 2004

(51) Int. Cl. A01K 67/04

(52) U.S. Cl. 119/270

(57) ABSTRACT

A metamorphosizing gift item in the form of a silk worm display and a method of gift-giving which utilizes the metamorphosis of silkworms into moths as representative of transformation in the gift-recipient's life. The silkworm display includes an open-ended transparent container closeable using a perforated lid. A silkworm cocoon spun on a twig and a plurality of artificial plants are anchored to a foam block affixed to the bottom inside of the container. The block is covered with peat moss to provide a realistic natural setting for the cocoon. The gift item is placed in a prominent location to allow observation in the morning upon rising from bed and before retiring to bed of the metamorphosis of the silkworm in the cocoon into a moth and the emergence from the cocoon. The metamorphosis symbolizes transformation in the gift-recipient's life such as a wedding, birth, recovery from illness, new job, and the like.

### BACKGROUND OF THE INVENTION

1. Field

The present invention relates generally to gift items and methods of gift-giving, and more particularly to gifts to for captive viewing of live plants, insects, and animals, and gift items made from or which include larval worms, cocoons, and butterflies or moths, and methods of gift-giving related thereto.

2. State of the Art

Greeting cards, flowers, money, and various other gift items have been given from one person to another on special occasions for centuries. Such special occasions include birthdays, holidays, graduation, retirement, illness, and the like. Such gift items acknowledge the occasion, tell the recipient that the giver cares, commemorate the occasion, and make the recipient feel good. The latter is especially important when the recipient is ill.

While greeting cards, flowers, money, and various other gift items are good for the purpose they serve, the only one listed which typically contains a message to the recipient is greeting cards. While greeting cards often accompany the other gift items, this is not always the case. Yet the message is typically the most important part of a gift item to the recipient. After the message is read, the greeting card is replaced into the envelope in which it came, or more often is placed in a prominent location where the recipient can view the greeting card. The experience of reading the greeting card is brief, typically lasting less than one minute. The message doesn't change in the greeting card, the special occasion passes, and consequently attention to the greeting card and its message quickly wains. Likewise, since greeting cards are given so often, the specialness thereof tends to wear thin over a person's lifetime due to the volumes of greeting cards received. There is nothing more to anticipate from the gift items once they are unwrapped and opened, or the message is read. Likewise, natural flowers wilt over a period of days or maybe a few weeks. Money is typically spent on products or services, the special occasion on which the money was given which more often than not is subsequently forgotten.

Therefore, greeting cards, flowers, money, and other gift items lack longevity of remembering the special occasion and the particular message. The experience of reading the greeting card or enjoying flowers is relatively brief. Such gift items and accompanying messages are given so often as to become much less memorable. The messages in greeting cards are often similar and thus less memorable.

Terrariums are sometimes given as gift items and have been used as decorative devices in homes for many years to permit convenient captive viewing of plants, insects, and reptiles, snakes, or other such animals. Some terrariums are made from converted aquariums which are partially filled with dirt, various plants are planted in the dirt, and a screen cover and/or lighting fixture are placed on top of the aquarium. If reptiles, snakes, or other animals are contained within the terrarium, food and water are also provided, and the screen cover or lighting fixture are of a type which keep the animal contained within the terrarium. The screen cover allows air to flow through the terrarium for ventilation and providing fresh air for breathing to the plants, insects, or animals contained therein.

Various containing devices have also been designed specifically for use as terrariums. Such terrariums may be of virtually any desired configuration of shape and size. Such configurations include cylindrical, globular, pyramidal, more complex shapes, and the like with any combination of side shapes such as rectangular, triangular, and the like.

Various terrariums and similar devices have been patented over the years. For example, in U.S. Pat. No. 5,404,839 issued to Mancuso on Apr. 11, 1995 is disclosed a terrarium having a glass side wall serving as a viewing window, and a chassis formed of rigid plastic providing a bottom wall and a rear side wall. In one version, the chassis includes a bottom channel formed in the bottom wall of the chassis for mounting the glass viewing wall. In another version, the glass wall is mounted to the chassis using a plastic frame member bonded to the plastic sidewalls of the chassis.

The terrarium also includes a top screened lid.

In U.S. Pat. No. 5,398,642 issued to Harwich on Mar. 21, 1995 is disclosed a terrarium specifically designed as an observation and feeding device for crawling insects such as ants. The device includes a central core member contained within a protective outer enclosure. The central core member extends within the enclosure between a base portion and an entrance portion and includes predetermined surfaces which define a plurality of levels. Some of the levels are used for holding food and other levels include ladders, mazes and the like for enhancing the insect observation experience.

Terrariums require care such as periodic of the plants, feeding and watering of the insects or animals, cleaning, and the like. Like greeting cards, flowers, money, and other gift items, though possibly to a lesser degree due to their more unique nature and less frequent giving to recipients, terrariums lack longevity of remembering the special occasion.

Silkworms which spin cocoons of silk prior to metamorphosizing into silkworm moths have been highly valued for centuries as a source of silk. The cocoons are unwrapped into silk strands which are processed, dyed, and woven into a variety of beautiful clothing items such as scarves. The cocoons themselves have also been used as decorative items which could presumably be given as gift items, as reflected in various patented devices and methods. For example, in U.S. Pat. No. 3,994,259 issued to Sakamura on Nov. 30, 1976 is disclosed a method of making cocoons with a sounding object. During the cocoon spinning stage of the silkworm a sounding object such as a bell is inserted into the cocoon. The method produces a product for use in folk handicrafts, personal dress accessories, and as a decorative accessory. Dye may be applied to the silkworm during cocoon spinning to color the silk as excreted by the silkworm to produce colored products.

Cocoons and silk spun by silkworms, and clothing, folk handicrafts, personal dress accessories, and decorative accessories made therefrom lack longevity of remembering the special occasion, though like terrariums, possibly to a lesser degree than greeting cards, flowers, money, and other gift items due to their more unique nature and less frequent giving to recipients.

There is a need for a gift item which: 1) is totally unique in comparison to other gift items; 2) provides a more memorable message; 3) teaches the recipient a valuable lesson; 4) provides a longer lasting experience than greeting cards and flowers; 5) provides longevity of remembering the particular special occasion; 6) provides a message which is memorable for the remainder of the recipient's life; and 7) requires little or no care.

### SUMMARY OF THE INVENTION

The present invention is a metamorphosizing gift item and a method of gift-giving which utilizes the life-cycle of a living being which is analogous to and representative of transformation in peoples' lives.

The gift item includes a container having an internal chamber, at least a portion of the container being transparent which permits outside observation thereinto. A metamorphosizing living being is disposed within the internal chamber which metamorphosizes during a portion of it's life-cycle while disposed within the container from a first state to a second state. The gift item is adapted to be placed in a location to allow observation of the development of the living being from the first to the second state, the metamorphosizing symbolizing transformation in observing peoples' lives.

In a preferred gift item, the container is a wall which encloses the internal chamber. The internal chamber has an open end which terminates at an access opening. At least a portion of the wall is the transparent portion of the container. The living being is a larval worm housed in a spun cocoon which undergoes the metamorphosis therein and emerges as a winged insect, either a moth or a butterfly. A closure member is adapted to removably cover the access opening to retain the living being therein. The closure member is perforated to permit ambient air to circulate within the internal chamber of the container while retaining the living being therein. At least one decorative item in the form of flowering plants, leafed plants, or ground cover are arranged in the container so as to provide a natural looking setting for the living being. A support block is affixed within the container adapted to anchor the decorative item there within. The gift item is adapted to be placed in a location to allow observation of the development of the living being from the first to the second state, the metamorphosizing symbolizing transformation in observing peoples' lives.

The method of gift-giving includes the step of providing a metamorphosizing gift item which includes a container having an internal chamber. At least a portion of the container is transparent which permits outside observation thereinto. A metamorphosizing living being is disposed within the internal chamber which metamorphosizes during a portion of it's life-cycle while disposed within the container from a first state to a second state. The metamorphosizing symbolizes transformation in observing peoples' lives.

In a first preferred method, the living being is the larval worm housed in the spun cocoon which undergoes the metamorphosis therein and emerges as the winged insect, either a moth or a butterfly. A further step includes disposing of the gift item in a manner by selling the gift item, giving the gift item to another person for a special occasion, or keeping the gift item for one's self.

A second preferred method further includes the steps of periodically observing the gift item to track development of the living being from the first to the second state, and placing the gift item in a location to allow observation of the development of the living being from the first to the second state. The living being is the larval worm housed in the spun cocoon which undergoes the metamorphosis therein and emerges as the winged insect, either a moth or a butterfly. The location in which the gift item is placed is at least seen in the morning upon rising from bed and before retiring to bed.

### THE DRAWINGS

The best mode presently contemplated for carrying out the invention is illustrated in the accompanying drawings, in which:

FIG. 1 is a front elevational view of a metamorphosizing gift item in accordance with the present invention in the form of a silkworm display designed to allow observation of the development and emergence of a moth from a cocoon disposed therein, which includes a transparent generally spherical container supported on a support base, a removable lid closing an access opening of the container, and shown supported on a counter top which is shown in lateral cross-section:

FIG. 2, a top plan view of the silkworm display to a smaller scale taken on the line 2-2 of FIG. 1 showing the construction of the lid which includes a flange ring with a hole covered by a netting, and a decorative ribbon tied in a bow;

FIG. 3, a bottom plan view of the container to the smaller scale showing a pair of oppositely disposed retaining tabs which removably retain the container to the base;

FIG. 4, a perspective view of the base to the smaller scale showing a pair of oppositely disposed tab receiving holes for receiving the retaining tabs of the container;

FIG. 5, a fragmentary longitudinal sectional view of the base to about the scale of FIG. 1 taken on the line 5-5 of FIG. 4;

FIG. 6, an exploded perspective view of the lid to the smaller scale showing the flange ring, the netting, and a plurality of attachment devices which removably connect the netting to the flange ring covering the hole; and

FIG. 7, a perspective view of a support block to about the scale of FIG. 1 with a plurality of decorative items extending upwardly therefrom, and a cocoon assembly which includes a silkworm cocoon spun on a stick also extending upwardly from the support block prior to placement within the container.

### DETAILED DESCRIPTION OF THE ILLUSTRATED EMBODIMENTS

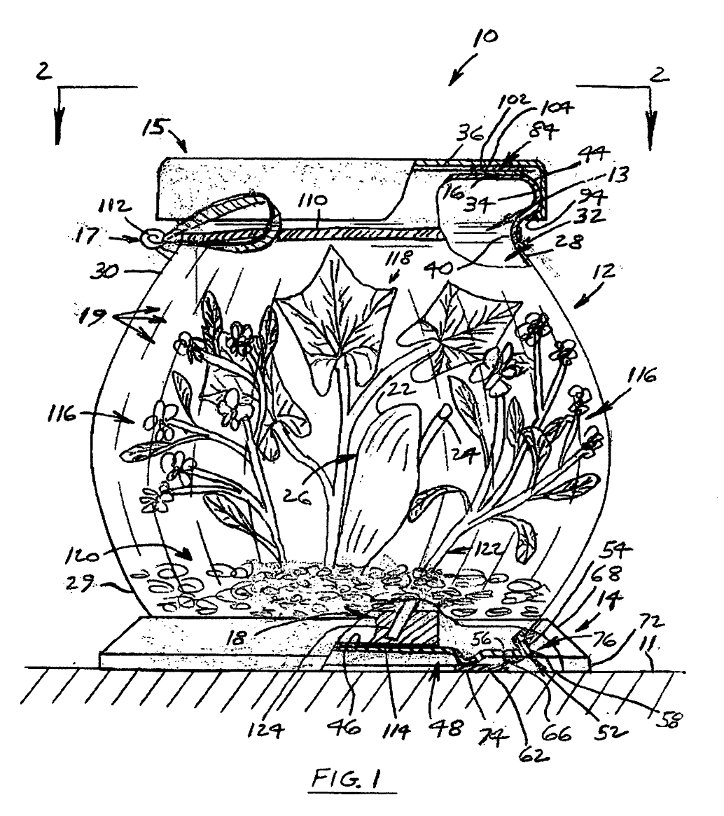

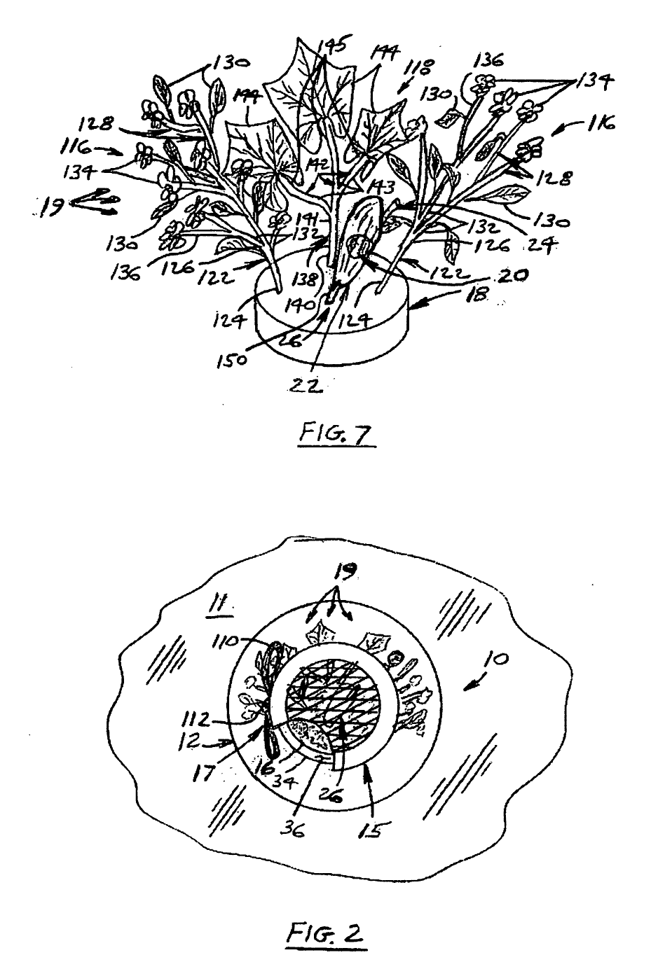

In FIGS. 1 and 2 is shown a metamorphosizing gift item in accordance with the present invention in the form of a silkworm display, designated generally at 10, on a support surface in the form of a counter top 11.

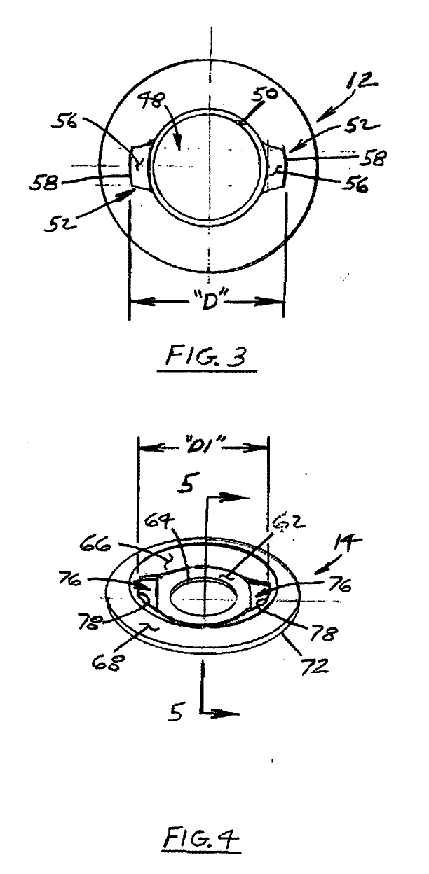

As best shown in FIGS. 1-3, the silkworm display 10 includes a transparent, generally spherical container 12 with an internal chamber 13. An annular support base 14 supports the container 12 on the counter top 11. A closure member in the form of a cap or lid 15 removably fits to the container 12 over an access opening 16 thereof. A decorative ribbon 17 is tied around container 12, being shown rotated horizontally ninety degrees from the front to better show other features of the silkworm display 10. A support block 18 affixed within container 12 anchors a plurality of decorative items 19 within the internal chamber 13.

The silkworm display 10 is for placement in a location to allow observation of the development and metamorphosizing of a living being contained therein from a first state to a second state, the metamorphosizing symbolizing transformation in the lives of observing people. The metamorphosizing living being is disposed within the internal chamber 13 among the decorative items 19. The living being is preferably a larval worm 20 housed in a cocoon 22 it spun around a twig or stick 24 and which is also anchored to the support block 18. The larval worm 20 undergoes metamorphosis during the portion of its life-cycle in the cocoon 22 disposed within the container 12 from a first state as the larval worm 20 and emerges in a second state as a winged insect in the form of a moth or butterfly (not shown). The cocoon 22 containing the larval worm 20 and the stick 24 comprise a cocoon assembly 26 which may be purchased as such.

The container 12 includes a transparent wall 28 of plastic or glass which encloses the internal chamber 13. While the container 12 shown is completely transparent, only a portion of the container 12 may be transparent if do desired which still permits adequate outside observation thereinto. The container 12 has a closed lower end 29 for retaining the decorative items 19 and the cocoon assembly 26 within the internal chamber 13. The access opening 16 is disposed at an open upper end 30 of the container 12, the internal chamber 13 having an open end 32 which terminates at the access opening 16. The container 12 is formed from a transparent material which permits unobstructed observation of the decorative items 19 and the cocoon assembly 26 from all angles when viewed from the outside. Alternatively, the container 12 may be transparent at one or more selected locations and translucent or opaque at other selected locations, and/or of an alternative shape such as rectangular, cylindrical, polygonal, and the like. At least a portion of the wall 28 is the transparent portion of the container 12.

The upper end 30 of container 12 has a radially outwardly dependent lid-receiving flange 34 which defines a flat upper surface 36 of container 12 with the access opening 16 which is closeable using the lid 15. A radially inwardly disposed neck 40 disposed below the access opening 16 is of a minimal inner diameter equal or slightly greater than access opening 16. The flange 34 has an annular rounded outer surface 44. A flat inside bottom 46 of container 12 is defined by an upwardly disposed recess 48 forming an annular horizontal support surface 50 which rests on base 14. A pair of retaining tabs 52 extend radially outwardly of support surface 50 disposed radially oppositely one another, including respective angled surfaces 54, flat bottom surfaces 56 disposed slightly above support surface 50, and rounded end surfaces 58 disposed at a diameter “D”.

As best shown in FIGS. 4 and 5, the base 14 is molded from a thin sheet 60 of plastic for engaging and supporting the container 12 on counter top 11. The base 14 may be of a construction integrally formed with the container 12 or as a separate piece as shown. The base 14 is shown as molded from a sheet 60 with a flat horizontal inner ring 62 with a central hole 64 therethrough, inner ring 62 being of a size a sufficient to receive and support container 12. An upwardly angled middle ring 66 is radially outwardly dependent from inner ring 62, and an downwardly angled outer ring 68 is radially outwardly dependent from middle ring 66 terminating at a downwardly dependent annular support rim 72. The inner ring 62 and support rim 72 define a horizontal support surface 74 which rests on the counter top 11. A pair of tab receiving holes 76 of substantially polygonal shape are disposed through the inner ring 62 and middle ring 66 radially opposite one another, each being of a configuration to closely receive retaining tabs 52. Respective end surfaces 78 of base 14 at holes 76 are at a diameter “D1” which is slightly less than diameter “D” such that base 14 snap-fits to container 12 to be removably attachable together.

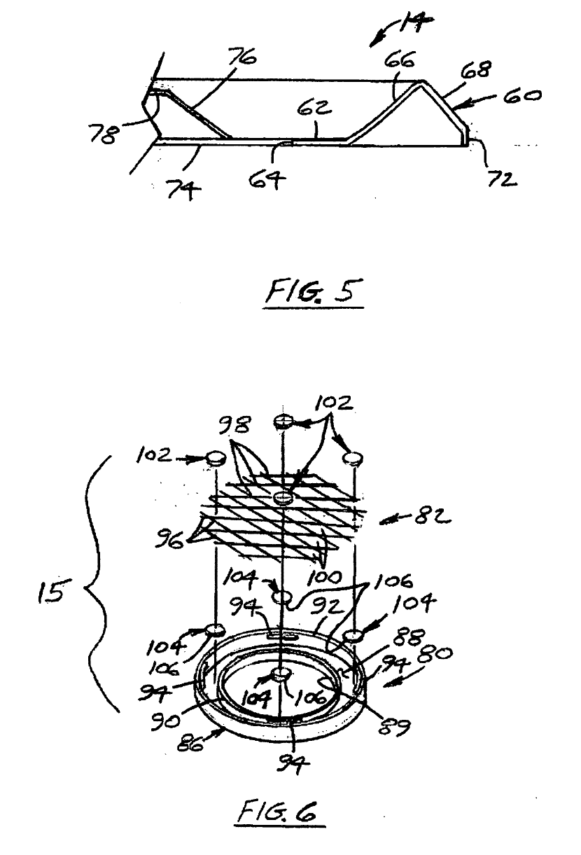

Referring to FIGS. 2 and 6, the lid 15 removably covers the access opening 16 to retain the living being therein. The lid 15 is perforated to permit ambient air to circulate within the internal chamber 13 of the container 12 while retaining the living being therein. The lid 15 includes a flange ring 80 which engages the flange 34 of container 12, a mesh or netting 82 which permits ambient air to circulate within the internal chamber 13 of container 12, and a plurality of attachment devices 84 removably peripherally connect netting 82 to flange ring 80 stretched horizontally thereacross.

The flange ring 80 is molded made from a sheet 86 of plastic in a circular configuration.

Flange ring 80 includes a flat horizontal base ring 88 which defines a hole 89 therethrough, a downwardly dependent annular inner leg or rim 90, and a downwardly dependent annular outer leg or rim 92. Outer rim 92 closely radially fits outside the outer surface 44 of flange 34. A plurality of retaining tabs 94 extend radially inwardly from outer rim 92. Flange ring 80 is sufficiently flexible to allow retaining tabs 94 to slide past outer rim 92 for application of lid 15 to container 12 in a secure manner while enabling the lid 15 to be readily removed from container 12 when required. The inner rim 90 fits within access opening 16 of container 12.

The container 12, base 14, and flange ring 80 are all typically constructed of a plastic material such as polyethylene, polypropylene, polyvinyl chloride, polycarbonate, or poly (methylmethacrylate), polystyrene resin, or a glass material. Other suitable transparent thermoplastic or thermoset materials may also be used. The container 12 is typically completely transparent, being formed by blow molding, injection molding, or other suitable process. The base 14 and flange ring 80 may be transparent, translucent, or opaque, typically being injection molded, vacuum formed and trimmed from sheet plastic, or other suitable process. Preferably, base 14 and flange ring 80 are formed in a circular configuration though alternative shapes such as rectangular, cylindrical, polygonal, and the like may be used. Other materials such as natural or stained wood may be used for the base 14 and flange ring 80 if so desired.

The netting 82 is formed in generally circular configuration to fit the hole 89 of flange ring 80. The netting 82 has a plurality of plastic filaments 96 with respective linear portions 98 of filaments 96 joined together to form a plurality of openings 100. The openings 100 are of a size between about one-quarter inch and one-half inch in length and width to retain the emergent moth or butterfly within the container 12. The netting 82 permits ambient air to circulate through lid 15 to be breathed by the larval worm 20 within the cocoon 22, and after metamorphosis by the emergent moth or butterfly. The netting 82 is of sufficient strength to retain the emergent moth or butterfly within container 12.

The netting 82 is typically conventional flexible plastic netting made of a transparent, translucent, or opaque plastic material such as polyethylene, polypropylene, or polyvinyl chloride material. Netting 82 is typically be formed by heat fusing, injection molding, or other suitable process. Other suitable thermoplastic or thermoset materials may also be used to form netting 82. Alternatively, less flexible conventional metal, plastic, or similar sheet mesh or screen materials may be used. Alternative shapes for netting 82 include rectangular, cylindrical, polygonal, and the like.

The attachment devices 84 comprise respective hook and loop disks 102 and 104 of removably interconnectable hook and loop fastener material commonly known as VELCRO™, loop disks 104 having respective rear adhesive layers 106 initially covered by respective removable protective cover sheets (not shown). Upon removal of the cover sheets, the loop disks 104 are adhesively affixed to flange ring 80 between the inner and outer rims 90 and 92 thereof. The netting 82 is then slightly stretched to form the openings 100, placed on loop disks 104 covering hole 89 of flange ring 80, and the hook disks 102 pressed thereover onto the mating loop disks 104 to sandwich and retain netting 82 therebetween. Alternatively, the netting 82 may be permanently bonded to flange ring 80 using conventional adhesives such as hot melt glue, cyanoacrylate adhesives, epoxies, acrylic adhesives, and other thermosetting adhesives. Plastic bonding processes such as heat fusing, ultrasonic welding, and the like may likewise be used.

Referring to FIGS. 1 and 2, the decorative ribbon 17 comprises a strip 110 of conventional ribbon material which is wrapped around the neck 40 of container 12 and tied in a bow 112. Rope or other decorative materials may be used in addition to or in place of ribbon 17.

As best shown in FIG. 7, the support block 18 is of a disk-like configuration, being made of a foamed synthetic resin or other porous plastic material such as polyethylene foam. The decorative items 19 and cocoon assembly 26 may be anchored thereto by inserting into the support block 18 so as to extend outwardly therefrom and anchor the decorative items 19 as done with conventional floral arrangements. The support block 18 is of as size as to fit onto the inside bottom 46 of container 12. Support block 18 is adhesively affixed to the inside bottom 46 of container 12 using an adhesive layer 114 comprised of a liquid adhesive soaked paper towel. Double sided adhesive tape may also be used if so desired. The inside bottom 46 may include a centrally disposed socket (not shown) for seating the support block 18 within container 12. Modeling clay or other such material may be used in place of the support block 18 and adhesive layer 114 to anchor the decorative items 19 and cocoon assembly 26 within container 12.

The decorative items 19 include artificial flowering plants 116, an artificial leafed plant 118, and ground cover in the form of decorator peat moss 120. The decorative items 19 are arranged in the container 12 so as to provide a natural looking setting for the living being.

The flowering plants 116 include respective main stems 122 which have bottom ends 124 that are inserted into the support block 18 and top ends 126 from which a plurality of upper stems 128 extend. A plurality of leaves 130 extend upwardly from main stems 122 at respective lower ends 132 thereof and a plurality of flowers 134 are attached to respective upper ends 136 thereof.

The leafed plant 118 includes a main stem 138 with a bottom end 140 that is inserted into the support block 18 and a top end 141 from which a plurality of upper stems 142 extend at respective lower ends 143. Respective leaves 144 extend from respective upper ends 145 of upper stems 142.

The flowering plants 116 and leafed plant 118 may simulate any desired type to provide a natural-looking backdrop and environment within container 12 for the cocoon assembly 26. Mulberry and edelberry plants are particularly appropriate for silk worms as these are the natural food and place of cocooning for silk worms.

The flowering plants 116 and leafed plant 118 are assembled to the support block 18 by applying liquid adhesive to the bottom ends 124 of the flowering plants 116 and to the bottom end 140 of the leafed plant 118. The bottom ends 124 and 140 are then inserted into the support block 18 so as to extend upwardly therefrom in a naturally appearing manner. The leafed plant 118 is shown centrally and slightly rearwardly disposed between the flowering plants 116 to with the cocoon assembly 26 centrally disposed on support block 18. The flowering plants 116 and the leafed plant 118 form a natural-looking backdrop for the cocoon assembly 26.

The flowering plants 116 and leafed plant 118 are constructed in conventional manner typically as a plurality of individually molded plastic component parts which snap-fit together. Alternatively, they may be integrally molded. The plastic is typically opaque or translucent such as polyethylene, polypropylene, or polyvinyl chloride which may be supported by being injection molded or otherwise molded about internal reinforcing wires (not shown) to allow bending and retaining in desired positions. Other suitable thermoplastic or thermoset materials may also be used. The flowering plants 116 and leafed plant 118 may also be of the commercially available silk construction.

Referring to FIG. 1, the peat moss 120 is added to the internal chamber 13 of container 12 to cover the support block 18 and provide a natural-looking ground surface for the silkworm display 10. The support block 18 is covered with the peat moss 120, which may be coated with a spray adhesive prior to insertion into container 12 to retain the peat moss 120 to support block 18. Other ground coverings may be used instead of or additionally to peat moss 120 such as gravel, sand, dirt, and combinations thereof.

As bet shown in FIG. 7, the cocoon assembly 26 includes the cocoon 22 spun about the stick 24 having a bottom end 150 which is inserted into the support block 18. The cocoon assembly 26 is centrally placed on the support block 18 by applying the liquid adhesive to the bottom end 150 of stick 24 and inserting the bottom end 150 into the support block 18. The cocoon 22 attached to the stick 24 is thus centrally positioned within container 12 with the peat moss 120 forming the natural looking ground surface with the flowering plants 116 and leafed plant 118 forming the natural-looking backdrop for the cocoon 22. The cocoon 22 houses the larval worm 20, preferably a Cecropia silkworm which metamorphoses therein from a rather mundane looking worm into a spectacular Cecropia silkworm moth, thus symbolizing the beneficial transformation in people's life. The cocoon assemblies 26 including the Cecropia silkworm moth cocoon 22 are commercially available such as from the Carolina Biological Supply Company of Burlington, N.C. Cecropia silkworm moth cocoons 22 are most readily available on a seasonal basis in the months of March through August.

The Cecropia silkworm moth is the largest insect in North America. People report that seeing the moth is a miraculous and incredible display of nature's beauty which will not soon be forgotten. That is why it is preferred for the silkworm display device 10 and a method of gift-giving, even though other butterflies and moths undergo similar metamorphoses. Alternatively, other types of cocoons 22 with other types of larval worms 20 may be used which emerge as moths or butterflies.

The method of gift-giving in accordance with the present invention utilizes the life-cycle of a living being, preferably the Cecropia silkworm moth, which is analogous to and representative of transformation in peoples' lives. The life-cycle of Cecropia silkworm moths is one of transformation and metamorphosis in which relatively non-enchanting and non-beautiful Cecropia silkworms form a silk cocoon around themselves only to emerge weeks later as the ever enchanting and beautiful Cecropia silkworm moths. Therefore, the following describes the Cecropia silkworm moth and life-cycle thereof.

The Cecropia moth begins life as an egg laid by a parent Cecropia moth in the fall season. The egg passes through the winter season and hatches as a larvae or silkworm in April or May of the following year. The newly hatched caterpillar or silkworm is covered with black hair. The silkworm goes through several stages of eating mulberry leaves and growing rapidly over a four week period, molting as it grows by casting off old outer skins about four times during the period. The silkworm ends each stage by stopping the eating mulberry leaves and goes into an inactive or dormant state of from one to two days, commonly called sleeps. About twenty-five days after the hatching from the egg, the now fully-grown silkworm permanently stops eating the mulberry leaves and shrinks slightly, becoming transparent light brown in color. The adult silkworm then spins a cocoon over a period of about two to five days which completely envelopes itself. The silkworm casts off its old skin and becomes a pupa in the now completed cocoon. In about ten to fifteen days, the pupa cast off its skin and becomes the Cecropia moth. The moth emerges from the cocoon in the early morning hours and the male moths copulate with the female moths. The female moths lay eggs starting in the evening of the same day they emerged from the cocoon and complete laying of the eggs the next morning. The die a short time later and the cycle starts over.

The life journey of the Cecopria moth is analogous to that of people, each having challenges which must be overcome in order to achieve one's place in life. For the silkworm, the journey begins in childhood as the silkworm and extends in adulthood as the moth. The silkworm has numerous predators such as bats, birds, bees, spiders, and many mammals. As silkworms, their sole purposes are to eat, grow, and survive. If the silk worm survives the childhood stage, the transition period is reached when metamorphosis from childhood to adulthood takes place. Now as an adult Cecoppia moth, its purpose now is to mate or breed and continue the life cycle or circle of life. The life journey of people is analogous. Everyone has various challenges in life, some the same and many differing from person-to-person depending on individual circumstances, many of which must overcome to survive in a similar manner to the Cecopria moth. These may include in childhood illnesses, bullies, accidents, learning disabilities, and the like. If people survive to reach adulthood, they likely have blossomed in personality, education, career, and other accomplishments. They then form families, have children, and raise those children to themselves blossom into adulthood, thus completing the circle of life like the Cecopria moth. While some people don't make it, enough do to perpetuate the species.

The silkworm display is used by placing in the home of a person who is undergoing a difficult period in their life such as an illness, or a positive life change such as marriage or having a baby, or other such metamorphosis in their life. The location of the silkworm display is that which is seen at least in the morning upon the person rising from bed and before retiring to bed. Therefore, the person is reminded at least twice per day of the plight of the Cecopria moth, that is the difficult journey from childhood to adulthood, and be inspired to fight on in the person's difficult situation or be inspired in the good changes in their life. The emergence of the Cecopria moth symbolizes surviving the difficult situation, a successful positive life change, accomplishment of one's goals, and the like.

The location of the silkworm display in the home is preferably relatively dry and away from heating or cooling vents to provide an even room temperature. The Cecropia moth emerges in between about two weeks to four months, depending on the age of the cocoon and environmental factors, transforming or metamorphosizing from childhood as the caterpillar or silkworm to adulthood as a beautiful Cecopria moth. When the Cecopria moth emerges from the cocoon, it quickly climbs to hang and start drying or inflating its wings. This process takes one to two days and the moth is not in an immediate hurry to fly away. After several days, the Cecopria moth may be released outdoors if the late spring, summer, or early fall season and the weather is warm enough. If the season is wrong or the weather too cold, the Cecopria moth may be kept in the container or let out to fly inside the house. Cecropia moths do not eat because they do not have a mouth or digestive system. They are calm creatures in fact they will hang on your clothes if you place them. If you place them there, however, their life term is only about six to fourteen.

The method of gift-giving which utilizes the life-cycle of a living being which is analogous to and representative of transformation in peoples' lives comprises the step of: providing a metamorphosizing gift item which includes a container having an internal chamber, at least a portion of the container being transparent which permits outside observation thereinto, a metamorphosizing living being disposed within the internal chamber which metamorphosizes during a portion of it's life-cycle while disposed within the container from a first state to a second state, the metamorphosizing symbolizing transformation in observing peoples' lives. The morphosizing living being is preferably the Cecopria moth due to the spectacular metamorphosis thereof from silkworm to moth.

The method may include one or more exemplary additional steps which include:
1. Moisturizing the cocoon prior to emergence of the winged insect by periodically dipping the cocoon in water, periodically misting the cocoon with water, providing a sponge member within the container which is periodically moistened with water to increase humidity therein, or combinations thereof. This step helps the cocoon to retain moisture to keep the metamorphosizing silkworm hydrated therewithin.
2. Storing the cocoon at a temperature of between about fifty to fifty-five degrees Fahrenheit prior to the living being disposed within the internal chamber to prevent premature emergence of the winged insect.
3. Placing the cocoon within the container proper side up for the winged insect to emerge. This is the same positioning as when the cocoon was spun and helps to assure proper metamorphosis and emergence of the moth.
4. Periodically observing the gift item to track development of the living being from the first to the second state.
5. Placing the gift item in a location to allow observation of the development of the living being from the first to the second state, the metamorphosizing symbolizing transformation in observing peoples' lives.
6. Sorting the living beings by sex and placing those of a desired sex within the container prior to the step of providing a metamorphosizing gift item. This may be done by weight or size, the males being larger and heavier than the females.
7. Selling the gift item, such as on-line, in flower shops, and the like.
8. Giving the gift item to another person for a special occasion such as Christmas, Easter, Thanksgiving, Labor Day, Fourth of July, Memorial Day, Valentines Day, New Home, Hanaka, New Years, Mother's Day, Confirmation, Barmishvas, Birthdays, Anniversaries, Communion, Get Well, New Job, Condolences, Graduation, Farewell, Sweetest Day, Father's Day, or Baby Showers. The gift item can be given as a replacement for or along with conventional flowers or a fruit basket.
9. Providing a written or recorded salutation for the gift item prior to giving the gift item to another person such as a conventional greeting card or small note card attached thereto.
10. Disposing of the living being by releasing outdoors, releasing indoors, or keeping in the container.

The method may include one or more exemplary variations which include:
1. The container provided in the step of providing a metamorphosizing gift item may have a base adapted to engage and support container on a support surface. The base may be of a construction of integrally formed with the container or a separate piece.
2. The gift item provided may have one or more decorative items such as flowering plants, leafed plants, and ground cover disposed within the internal chamber of the container arranged so as to provide a natural looking setting for the living being.
3. Any plants provided may be artificial, and any ground cover provided may be decorator peat moss, gravel, sand, dirt, or combinations thereof.
4. The living being provided may be a larval worm housed in a spun cocoon which undergoes the metamorphosis therein and emerges as a winged insect of a moth or butterfly.
5. The cocoon provided may be is spun about a twig.
6. The larval worm provided preferably is a Cecropia silkworm which metamorphoses into a Cecropia silkworm moth.
7. The location in which the gift item is placed may at least seen in the morning upon rising from bed and before retiring to bed.
8. Sorting of the living beings by sex may be done by a method of weighing, sizing, or a combination thereof.

In a first preferred method, the living being is the larval worm housed in the spun cocoon. The larval worm undergoes the metamorphosis therein and emerges as the winged insect, either a moth or a butterfly. A further step includes disposing of the gift item in a manner by selling the gift item, giving the gift item to another person for a special occasion, or keeping the gift item for one's, self.

A second preferred method further includes the steps of periodically observing the gift item to track development of the living being from the first to the second state, and placing the gift item in a location to allow observation of the development of the living being from the first to the second state. The living being is the larval worm housed in the spun cocoon which undergoes the metamorphosis therein and emerges as the winged insect, either a moth or a butterfly. The location in which the gift item is placed is at least seen in the morning upon rising from bed and before retiring to bed.

The gift item and method of giving a gift item of the present invention therefore satisfy all of the above-enumerated objectives including: 1) being totally unique in comparison to other gift items; 2) providing a more memorable message than conventional gift items such as greeting cards; 3) teaches the recipient a valuable lesson; 4) provides a longer lasting experience than greeting cards and flowers; 5) provides longevity of remembering the particular special occasion; 6) provides a message which is memorable for the remainder of the recipient's life; and 7) requires little or no care.

Whereas this invention is here illustrated and described with reference to embodiments thereof presently contemplated as the best mode of carrying out such invention in actual practice, it is to be understood that various changes may be made in adapting the invention to different embodiments without departing from the broader inventive concepts disclosed herein and comprehended by the claims that follow.

1. A metamorphosizing gift item, comprising:
a container having an internal chamber, at least a portion of said container being transparent which permits outside observation thereinto;
a metamorphosizing living being disposed within said internal chamber which metamorphosizes during a portion of it's life-cycle while disposed within said container from a first state to a second state; and
wherein the gift item is adapted to be placed in a location to allow observation of the development of said living being from said first to said second state, said metamorphosizing symbolizing transformation in observing peoples' lives.

2. The gift item according to claim 1, wherein the container comprises a wall which encloses the internal chamber, said internal chamber having an open end which terminates at an access opening, at least a portion of said wall being the transparent portion of said container, and further comprising a closure member adapted to removably cover said access opening to retain the living being therein.

3. The gift item according to claim 2, wherein the closure member is perforated to permit ambient air to circulate within the internal chamber of the container while retaining the living being therein.

4. The gift item according to claim 1, wherein the container has a closed lower end and the access opening is disposed at an open upper end of said container.

5. The gift item according to claim 1, wherein the container is of a general configuration chosen from the group consisting of spherical, rectangular, cylindrical, and polygonal.

6. The gift item according to claim 1, further comprising a base adapted to engage and support container on a support surface, said base being of a construction chosen from the group consisting of integrally formed with the container and a separate piece.

7. The gift item according to claim 1, further comprising at least one decorative item chosen from the group consisting of flowering plants, leafed plants, and ground cover, said decorative items being arranged in the container so as to provide a natural looking setting for the living being.

8. The gift item according to claim 7, wherein the plants are artificial.

9. The gift item according to claim 7, wherein the ground cover is chosen from the group consisting of decorator peat moss, gravel, sand, dirt, and combinations thereof.

10. The gift item according to claim 1, further comprising a support block affixed within the container adapted to anchor items therewithin.

11. A device as set forth in claim 10, wherein the support block comprises a foamed synthetic resin, the items being affixed thereto by inserting a portion thereof into said support block so as to extend outwardly therefrom.

12. The gift item according to claim 1, wherein the living being comprises a larval worm housed in a spun cocoon which undergoes the metamorphosis therein and emerges as a winged insect chosen from the group consisting of moths and butterflies.

13. The gift item according to claim 12, wherein the cocoon is spun about a twig.

14. The gift item according to claim 12, wherein the larval worm comprises a Cecropia silkworm which metamorphoses into a Cecropia silkworm moth.

15. A metamorphosizing gift item, comprising:
a container having an internal chamber, at least a portion of said container being transparent which permits outside observation thereinto, said container comprising a wall which encloses said internal chamber, said internal chamber having an open end which terminates at an access opening, at least a portion of said wall being the transparent portion of said container;
a metamorphosizing living being disposed within said internal chamber which metamorphosizes during a portion of it's life-cycle while disposed within said container from a first state to a second state, said living being comprising a larval worm housed in a spun cocoon which undergoes the metamorphosis therein and emerges as a winged insect chosen from the group consisting of moths and butterflies;
a closure member adapted to removably cover said access opening to retain the living being therein, said closure member being perforated to permit ambient air to circulate within said internal chamber of said container while retaining said living being therein;
at least one decorative item chosen from the group consisting of flowering plants, leafed plants, and ground cover, said decorative items being arranged in said container so as to provide a natural looking setting for said living being;
a support block affixed within the container adapted to anchor said decorative item therewithin; and
wherein the gift item is adapted to be placed in a location to allow observation of the development of said living being from said first to said second state, said metamorphosizing symbolizing transformation in observing peoples' lives.

16. The gift item according to claim 15, wherein the larval worm comprises a Cecropia silkworm which metamorphoses into a Cecropia silkworm moth.

17. The gift item according to claim 16, wherein the cocoon is spun about a twig.

18. The gift item according to claim 16, further comprising:
a base adapted to engage and support container on a support surface, said base being of a construction chosen from the group consisting of integrally formed with the container and a separate piece; and
wherein said container has a closed lower end and the access opening is disposed at an open upper end of said container, said container being of a general configuration chosen from the group consisting of spherical, rectangular, cylindrical, and polygonal, wherein the plants are artificial and the ground cover is chosen from the group consisting of decorator peat moss, gravel, sand, dirt, and combinations thereof, and wherein the support block comprises a foamed synthetic resin, the items being affixed thereto by inserting a portion thereof into said support block so as to extend outwardly therefrom.

19. A method of gift-giving which utilizes the life-cycle of a living being which is analogous to and representative of transformation in peoples' lives, comprising the step of:
providing a metamorphosizing gift item which includes a container having an internal chamber, at least a portion of the container being transparent which permits outside observation thereinto, a metamorphosizing living being disposed within the internal chamber which metamorphosizes during a portion of it's life-cycle while disposed within the container from a first state to a second state, the metamorphosizing symbolizing transformation in observing peoples' lives.

20. The method according to claim 19, wherein the container provided in the step of providing a metamorphosizing gift item has a base adapted to engage and support container on a support surface, the base being of a construction chosen from the group consisting of integrally formed with the container and a separate piece.

21. The method according to claim 19, wherein the gift item provided has at least one decorative item chosen from the group consisting of flowering plants, leafed plants, and ground cover disposed within the internal chamber of the container arranged so as to provide a natural looking setting for the living being.

22. The method according to claim 21, wherein any plants provided are artificial, and any ground cover provided is chosen from the group consisting of decorator peat moss, gravel, sand, dirt, and combinations thereof.

23. The method according to claim 19, wherein the living being provided comprises a larval worm housed in a spun cocoon which undergoes the metamorphosis therein and emerges as a winged insect chosen from the group consisting of moths and butterflies.

24. The method according to claim 23, wherein the cocoon provided is spun about a twig.

25. The method according to claim 23, wherein the larval worm provided comprises a Cecropia silkworm which metamorphoses into a Cecropia silkworm moth.

26. The method according to claim 23, further comprising the step of moisturizing the cocoon prior to emergence of the winged insect using a method chosen from the group consisting of periodically dipping the cocoon in water, periodically misting the cocoon with water, providing a sponge member within the container which is periodically moistened with water to increase humidity therein, and combinations thereof.

27. The method according to claim 23, further comprising the step of storing the cocoon at a temperature of between about 50 to 55 degrees Fahrenheit prior to the living being disposed within the internal chamber to prevent premature emergence of the winged insect.

28. The method according to claim 23, further comprising the step of placing the cocoon within the container proper side up for the winged insect to emerge.

29. The method according to claim 19, further comprising the step of periodically observing the gift item to track development of the living being from the first to the second state.

30. The method according to claim 19, further comprising the step of placing the gilt item in a location to allow observation of the development of the living being from the first to the second state, the metamorphosizing symbolizing transformation in observing peoples' lives.

31. The method according to claim 30, wherein the location in which the gift item is placed is at least seen in the morning upon rising from bed and before retiring to bed.

32. The method according to claim 19, further comprising the step of sorting the living beings by sex and placing those of a desired sex within the container prior to the step of providing a metamorphosizing gift item.

33. The method according to claim 32, wherein sorting of the living beings by sex is done by a method chosen from the group consisting of weighing, sizing, and a combination thereof.

34. The method according to claim 19, further comprising the step of selling the gift item.

35. The method according to claim 19, further comprising the step of giving the gift item to another person for a special occasion.

36. The method according to claim 35, further comprising the step of providing a written or recorded salutation for the gift item prior to giving the gift item to another person.

37. The method according to claim 19, further comprising the step of disposing of the living being in a manner chosen from the group consisting of releasing outdoors, releasing indoors, and keeping in the container.

38. A method of gift-giving which utilizes the life-cycle of a living being which is analogous to and representative of transformation in peoples' lives, comprising the steps of:
providing a metamorphosizing gift item which includes a container having an internal chamber, at least a portion of the container being transparent which permits outside observation thereinto, a metamorphosizing living being comprising a larval worm housed in a spun cocoon disposed within the internal chamber which metamorphosizes therein during a portion of it's life-cycle while disposed within the container from a first state as said larval worm and emerges in a second state as a winged insect chosen from the group consisting of moths and butterflies, the metamorphosizing symbolizing transformation in observing peoples' lives; and
disposing of the gift item in a manner chosen from the group consisting of selling the gift item, giving the gift item to another person for a special occasion, and keeping the gift item for one's self.

39. The method according to claim 38, wherein the gift item provided has at least one decorative item chosen from the group consisting of flowering plants, leafed plants, and ground cover disposed within the internal chamber of the container arranged so as to provide a natural looking setting for the living being, wherein any plants provided are artificial, and any ground cover provided is chosen from the group consisting of decorator peat moss, gravel, sand, dirt, and combinations thereof, and wherein the larval worm provided comprises a Cecropia silkworm which metamorphoses into a Cecropia silkworm moth.

40. A method of gift-giving which utilizes the life-cycle of a living being which is analogous to and representative of transformation in peoples' lives, comprising the steps of:
providing a metamorphosizing gift item which includes a container having an internal chamber, at least a portion of the container being transparent which permits outside observation thereinto, a metamorphosizing living being disposed within the internal chamber which metamorphosizes during a portion of it's life-cycle while disposed within the container from a first state to a second state, the metamorphosizing symbolizing transformation in observing peoples' lives.
periodically observing the gift item to track development of the living being from the first to the second state;
placing the gift item in a location to allow observation of the development of the living being from the first to the second state, the metamorphosizing symbolizing transformation in observing peoples' lives; and
wherein the living being provided comprises a larval worm housed in a spun cocoon which undergoes the metamorphosis therein and emerges as a winged insect chosen from the group consisting of moths and butterflies, and wherein the location in which the gift item is placed is at least seen in the morning upon rising from bed and before retiring to bed.

41. The method according to claim 40, further comprising the step of:
further comprising the step of moisturizing the cocoon prior to emergence of the winged insect using a method chosen from the group consisting of periodically dipping the cocoon in water, periodically misting the cocoon with water, providing a sponge member within the container which is periodically moistened with water to increase humidity therein, and combinations thereof; and
wherein the gift item provided has at least one decorative item chosen from the group consisting of flowering plants, leafed plants, and ground cover disposed within the internal chamber of the container arranged so as to provide a natural looking setting for the living being, wherein any plants provided are artificial, and any ground cover provided is chosen from the group consisting of decorator peat moss, gravel, sand, dirt, and combinations thereof, and wherein the larval worm provided comprises a Cecropia silkworm which metamorphoses into a Cecropia silkworm moth.
